(19) United States
(12) Patent Application Publication
(10) Pub. No.: US 2007/0092572 A1
(43) Pub. Date: Apr. 26, 2007

(54) Bone cement compositions and the like comprising an RNAIII-inhibiting peptide

(76) Inventors: Naomi Balaban, Hopkinton, MA (US); Joel Braunstein, Pikesville, MD (US)

Correspondence Address: MERCHANT & GOULD PC, P.O. BOX 2903, MINNEAPOLIS, MN, 55402-0903, US

(21) Appl. No.: 11/480,825

(22) Filed: Jul. 6, 2006

Related U.S. Application Data
(63) Continuation-in-part of application No. 11/394,228, filed on Mar. 31, 2006.
(60) Provisional application No. 60/667,940, filed on Apr. 4, 2005.

(51) Int. Cl. A61K 38/18; A61K 38/14; A61K 31/7034; A61K 33/42; A61K 9/14

(52) U.S. Cl. 424/487; 106/690; 106/691; 424/602; 514/12; 514/8; 524/35

(57) ABSTRACT

RNAIII-inhibiting peptide (RIP) advantageously treats or reduces the risk of biofilm formation on implanted bone cement, thus reducing the possibility of sustained chemotherapy, hospitalization, or surgical removal of the bone cement. Avoiding sustained chemotherapy reduces the risk of inducing antibiotic resistance. Accordingly, RIP can be used with antibiotics that otherwise would be reserved as a last resort, such as vancomycin. RIP potentiates the activity of vancomycin, minimizing the risk of inducing vancomycin-resistance. The present invention thus is expected to prolong significantly the effectiveness of antibiotics of last resort for the treatment of periprosthetic infection.

### CROSS REFERENCE TO RELATED CASES

This application is a continuation-in-part of application Ser. No. 11/394,228, filed Mar. 31, 2006, which claims the benefit of Provisional U.S. Application Ser. No. 60/667,940, filed Apr. 4, 2005, each of which is incorporated by reference herein in its entirety.

### BACKGROUND

1 . Technical Field

This application relates generally to compositions and methods for treating bacterial infection, particularly to a bone cement composition comprising an RNAIII-inhibiting peptide and an antibiotic.

2. Background of the Technology

Quorum Sensing and RNAIII-Inhibiting Peptide

Recent studies have evidenced the importance of quorum sensing in the pathology of bacterial species, including Vibrio cholerae, Pseudomonas aeruginosa, and Staphylococcus aureus. Quorum sensing is a mechanism through which a bacterial population receives input from neighboring cells and elicits an appropriate response to enable itself to survive within the host. See Balaban et al., Science 280: 438-40 (1998); Miller et al., Cell 110: 303-14 (2002); Hentzer et al., EMBO J. 22: 3803-15 (2003); Korem et al., FEMS Microbiol. Lett. 223: 167-75 (2003). In Staphylococcus, quorum sensing controls the expression of proteins implicated in bacterial virulence, including colonization, dissemination, and production of multiple toxins involved in disease promotion. Virulence factors include enterotoxins and toxic-shock syndrome toxin-1 (TSST-1), which act as superantigens to over-stimulate the host immune system, causing excessive release of cytokines and inducing the hyper-proliferation of T cells.

In a quorum sensing system in S. aureus, the effector quorum-sensing molecule “RNAIII-activating peptide” (RAP) phosphorylates “target of RNAIII-activating protein” (TRAP). TRAP is a 21 kDa protein that is highly conserved among Staphylococci species. TRAP phosphorylation promotes bacterial adhesion and the downstream production of a regulatory RNA molecule termed RNAIII, which is responsible for toxin synthesis. Balaban (1998); Balaban et al., J. Biol. Chem. 276: 2658-67 (2001). An antagonist of RAP, “RNAIII-inhibiting peptide” (RIP), inhibits the phosphorylation of TRAP and thereby strongly inhibits the downstream production of virulence factors, bacterial adhesion, biofilm formation, and infections in vivo. The mechanism of action of RIP is different from common antibiotics: instead of killing bacteria, RIP inhibits bacterial cell-cell communication, rendering the bacteria more vulnerable to host defense mechanisms. See Balaban (1998); Balaban et al., Peptides 21: 1301-11 (2000); Gov et al., Peptides 22:1609-20 (2001); Balaban et al., J. Infect. Dis. 187: 625-30 (2003); Cirioni et al., Circulation 108: 767-71 (2003); Ribeiro et al., Peptides 24: 1829-36 (2003); Giacometti et al., Antimicrob. Agents Chemother. 47: 1979-83 (2003); Balaban et al., Kidney Int. 23: 340-45 (2003); Balaban et al., Antimicrob. Agents Chemother. 48: 2544-50 (2004); Dell Acqua et al., J. Infect. Dis. 190: 318-20 (2004).

Biofilm Infections of Bone Cement

Bone cement compositions are used to strengthen damaged bone or to fix an implant material, e.g., an artificial joint, to a bone stock. Such applications are particularly useful in the areas of orthopedics, dentistry and related medical disciplines. Typically, a surgeon prepares bone cement directly before surgery by mixing polymethylmethacrylate (PMMA) powder with a liquid component comprising methyl methacrylate and crystals of barium sulfate, which make the resulting product radio-opaque. The surgeon presses or injects the resulting settable fluid substance into a cavity in the bone, and the fluid typically polymerizes and hardens within minutes. Many commercial formulations of bone cement are available that differ in chemical composition and physical properties, and new means of mixing and injecting bone cement are currently being developed.

Bone cement surfaces often support colonization of bacteria, leading to postoperative infections; therefore, most bone cements contain admixed antibiotics that act as a prophylactic for postoperative infections. Admixed antibiotics typically are used in combination with systemic antibiotics. See Hallab et al., J. Bone Joint Surg. 83-A: 428-36 (2001). Bacteria colonizing bone cement surfaces are difficult to eradicate with conventional antibiotics due to the formation of a biofilm on the prosthetic surface. Biofilms consist of multiple layers of adhering bacteria embedded in a matrix of secreted, adhesive exopolymers composed mainly of polysaccharides, known as a “glycocalyx.” The resistance of periprosthetic infections to host defense mechanisms and to chemotherapy is largely related to the protective environment of the glycocalyx. See, e.g., Dobbins et al. (1988).

Although antibiotics reduce implant-associated biofilms, they are very difficult to eradicate. The continued presence of antibiotics around the implant, coupled with incomplete killing of the bacteria, increases the risk of inducing antibiotic-resistant strains. See Van de Belt et al., Acta Orthop. Scand. 71: 625-29 (2000). The Center for Disease Control estimates that annually in the United States 2 million patients contract nosocomial (i.e., hospital acquired) infections with an annual mortality of nearly 100,000 people; approximately 70% of bacteria responsible for these infections are resistant to at least one of the drugs most commonly used to treat such an infection. An estimated 70% of the 2 million cases are associated with indwelling medical devices, with two thirds of these infections being due to S. aureus and S. epidermidis. See Weinstein, “Nosocomial Infection Update,” Emerging Infectious Diseases 4: 416-20 (1998).

Postoperative infections after orthopedic surgery can have devastating consequences, both in terms of cost and preventable patient morbidity and mortality. Treatment options for implant-related infections vary but typically involve a combination of surgical debridement and systemic antibiotics. Infections involving implanted bone cement usually require weeks to months of intravenous antibiotic administration, bed confinement, immobility, and/or prosthesis extraction with shattering of nearby bone and destruction of surrounding soft tissue. While prolonged antibiotic exposure and bone cement extraction often are successful in eradicating the infection, recovery is suboptimal and often leaves patients with long-term functional impairment. Accordingly, there is an urgent need for an effective, safe and fast-acting drug to prevent and treat infections associated with implanted bone cement, especially with biofilm associated infections by drug resistant bacteria.

### SUMMARY

An RNAIII-inhibiting peptide (RIP) meets this need by inhibiting biofilm formation and toxin production in bacteria that colonize a bone cement implant and infect nearby tissue. Unlike antibiotics, RIP eradicates biofilms without inducing resistant bacterial strains. Treatment with RIP thus reduces the necessity for sustained chemotherapy, hospitalization, or surgical removal of the bone cement. Avoiding sustained chemotherapy in turn reduces the risk of inducing antibiotic resistance. Accordingly, RIP can be used with antibiotics that otherwise would be reserved as a last resort, such as vancomycin or daptomycin. RIP potentiates the activity of vancomycin, minimizing the risk of inducing vancomycin resistance. The present invention thus is expected to prolong significantly the effectiveness of antibiotics of last resort for the treatment of periprosthetic infection.

RIP may be administered in a number of ways. For example, RIP may be admixed with the bone cement composition before implanting. RIP itself may be combined in a burst release or sustained release formulation, e.g., nanoparticles comprising a RIP composition, which can be admixed or otherwise administered with the bone cement composition. Because RIP functions by a different mechanism than antibiotics, RIP can complement antibiotic efficacy. RIP accordingly can be used in combination with admixed or systemically delivered antimicrobial agents, such as an antibiotic or antimicrobial peptide. RIP also can be used with such agents as an anesthetic or bone morphogenetic protein.

According to a first embodiment, a bone cement composition comprises a RIP. The bone cement composition further may comprise an antibiotic (e.g., vancomycin), antimicrobial peptide, anesthetic, or bone morphogenic protein. The RIP may be present in an amount effective to treat or reduce the risk of biofilm formation on or around the bone cement implant. The RIP may be formulated with a carrier system capable of burst release or sustained release kinetics, which formulation may comprise nanoparticles. The present invention may be practiced with any type of bone cement composition, including those comprising polymethylmethacrylate or methyl methacrylate, and including injectable ceramic cements, injectable calcium phosphate hydraulic cements, calcium deficient hydroxyapatite cements, dahllite cements, or brushite cements.

According to a second embodiment, a method of administering a bone cement comprises co-administering a RIP composition, where the RIP composition may be added before, during or after addition of the bone cement. For example, RIP may be admixed with a powdered component of the bone cement or added to the bone cement prior to setting. The RIP composition may be administered parenterally or by any other suitable route. The RIP composition may further comprise an antimicrobial agent, such as an antibiotic or antimicrobial peptide, or an anesthetic or bone morphogenic protein. The administration of the bone cement composition comprising RIP may be repeated on the same individual, as necessary.

According to a third embodiment, a biodegradable composition comprises a RIP. The biodegradable composition may be a fibrin sealant. The fibrin sealant may be a surgical adhesive glue, surgical sealant, or the like. As with bone cement, the fibrin sealant may be manufactured or stored with admixed RIP while in powdered form or in any other pre-solidified or pre-implanted form. A RIP similarly may be added to biodegradable compositions like collagen sheet hydrogels or hydrocolloids or the like used for wound care. Hydrogels and hydrocolloids include collagen alginate wound dressings, temporary skin replacements and scar removal sheets.

### BRIEF DESCRIPTION OF THE DRAWINGS

FIG. 1 depicts the regulation of bacterial virulence via TRAP and agr.

FIG. 2 depicts a rat graft model system, which is a representative animal model useful for testing RIP compositions of the present invention.

FIG. 3 depicts the kinetics of the release of RIP (μg/mL) from a PMMA composition preloaded with 7.5% w/w RIP.

FIG. 4A depicts the effect of RIP mixed with a PMMA composition on the number of colony forming units(CFU)/mL of multi-drug resistant S. aureus in a mouse model system. The weight percent of RIP in each composition is indicated.

FIG. 4B compares the effect of RIP and/or vancomycin mixed with a PMMA composition on the CFU/mL of multi-drug resistant S. aureus in a mouse model system. The weight percent of RIP in each composition is indicated.

FIG. 5 depicts the results of another mouse study using a PMMA composition loaded with RIP and/or vancomycin at the indicated weight percentages.

### DETAILED DESCRIPTION

The present invention provides a bone cement composition comprising RIP, which advantageously treats or reduces the risk of biofilm formation associated with the implanted bone cement, thus preventing time consuming, expensive and possible painful chemotherapy and hospitalization and reducing the possibility that the bone cement would have to be surgically removed. RIP is particularly advantageous in this application because it treats or reduces the risk of biofilms that often form on the surface of bone cement implants or other biodegradable compositions. RIP has a further advantage in this application because, unlike antibiotics, prolonged exposure of bacteria to RIP generally does not induce resistant strains.

RNAIII-Inhibiting Peptides of the Invention

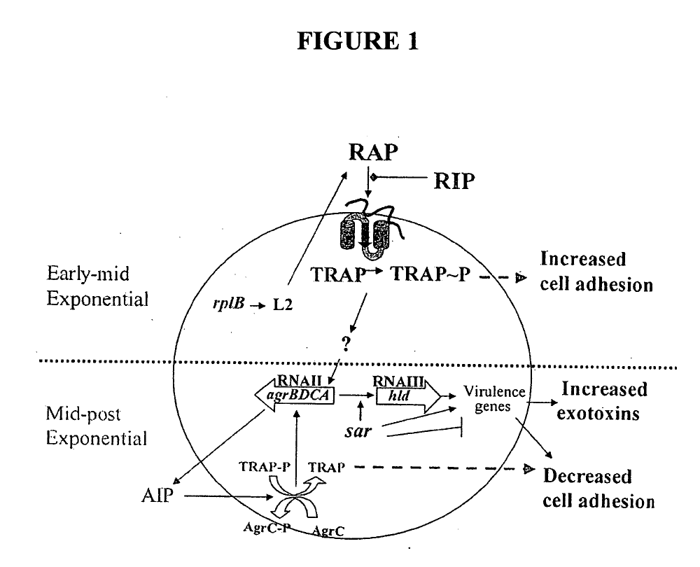

The quorum sensing inhibitor RIP does not affect bacterial growth but reduces the pathogenic potential of bacteria by interfering with the signal transduction that leads to production of exotoxins. RIP blocks toxin production by inhibiting the phosphorylation of its target molecule TRAP, which is an upstream activator of the agr locus. FIG. 1 depicts the role of TRAP phosphorylation in the downstream activation of the agr locus. As cells multiply, RAP accumulates in the extracellular milieu and promotes TRAP phosphorylation, leading to increased bacterial adhesion and agr activation in the mid-exponential stage of growth. Agr activation leads to the production of Autoinducing Peptide (AIP), which reduces TRAP phosphorylation but allows expression of RNAIII, which increases hemolysin and enterotoxin production. RIP or a RIP agonist, such as an anti-RAP antibody, inhibits TRAP phosphorylation, shifting the equilibrium to the non-phosphorylated, inactive form of the TRAP enzyme and blocking agr expression, thereby decreasing the adherence, biofilm formation, and toxin production of the bacteria.

RIP comprises the general formula YX₂PX₁TNF, where X₁ is C, W, I or a modified amino acid, and X₂ is K or S. Specific RIP sequences are disclosed in U.S. Pat. No. 6,291,431, application Ser. No. 10/358,448, filed Feb. 3, 2003, application Ser. No. 09/839,695, filed Apr. 19, 2001, and Gov et al., Peptides 22:1609-20 (2001), all of which are incorporated herein by reference. RIP sequences include polypeptides comprising the amino acid sequence KKYX₂PX₁TN, where X₁ is C, W, I or a modified amino acid and X₂ is K or S. RIP sequences also include polypeptides comprising YSPX₁TNF, where X₁ is C or W, and YKPITN. In one embodiment, the RIP comprising the general formula YX₂PX₁TNF above is further modified by one or two amino acid substitutions, deletions, and other modifications, provided the RIP exhibits activity.

The terms “protein,” “polypeptide,” or “peptide,” as used herein include modified sequences (e.g., glycosylated, PEG-ylated, containing conservative amino acid substitutions, containing protective groups, including 5-oxoprolyl, amidation, D-amino acids, etc.). Amino acid substitutions include conservative substitutions, which are typically within the following groups: glycine, alanine; valine, isoleucine, leucine; aspartic acid, glutamic acid; asparagine, glutamine; serine, threonine; lysine, arginine; and phenylalanine, tyrosine.

Proteins, polypeptides and peptides of the invention can be purified or isolated. “Purified” refers to a compound that is substantially free, e.g., about 60% free, about 75% free, or about 90% free, from components that normally accompany the compound as found in its native state. An “isolated” compound is in an environment different from that in which the compound naturally occurs. Proteins, polypeptides and peptides of the invention may be naturally occurring or produced recombinantly or by chemical synthesis according to methods well known in the art.

Bone Cement Compositions

Bone cements are used to fill in gaps in bones and strengthen injured bones, e.g., wrists, hips and spines. For example, bone cement may be used for patients who might otherwise require complex hip reconstruction for avascular necrosis (i.e., bone death), and bone cement may be used in combination with other surgical procedures, such as insertion of other types of implants, pins, staples, etc. Applications of bone cements are known to the artisan of skill in this area and include uses in dental surgery, bone surgery, cosmetic reconstruction, traumatology, interventional radiology, and rheumatology.

“Bone cement compositions,” as the term is used herein, include those compositions based on PMMA and methyl methacrylate, and the like. Other examples of bone cement compositions include injectable ceramics and injectable calcium phosphate hydraulic cements, such as calcium deficient hydroxyapatite cements, including coral-derived Pro Osteon hydroxyapatite, dahllite cements, or brushite cements. See, e.g., Hardouin et al., “New injectable composites for bone replacement,” Semin. Musculoskeletal Radiol. 1(2): 319-24 (1997). These compositions advantageously are biocompatible, resorbable, osteoconductive, and injectable, allowing delivery with a syringe and needle through a percutaneous approach. They may be used in combination with, or as an alternative to, non-resorbable bone cement compositions. See Betz, Orthopedics J. 25(5 Suppl.): S561-70 (2002). Bone cements also include adhesive acrylics, which may be used at the interface between metal stems and cured bone cement. Suitable adhesive acrylic bone cements contain 4-methacryloyloxyethyl trimellitate anhydride (4-META), which is applied as a coating material to increase the strength of the cemented fixation. See Morita et al., J. Biomed Mater. Res. 34: 171-75 (1997). “Bone graft compositions” also include dimineralized bone matrix, synthetic bone graft substitutes, cross-linked collagen bone grafts, bone graft putty and the like.

“Bone cement compositions” include bone cement formulations before and after insertion or implantation into an individual. That is, a RIP may be administered to an individual after the bone cement has solidified within the individual. Alternatively or additionally, the bone cement composition may be manufactured or stored with admixed RIP. Thus, in one embodiment, a component of the bone cement composition that comprises RIP may be in powdered form.

Biodegradable Compositions Comprising RIP

A RIP composition may be used to treat or reduce the risk of biofilms associated with other biodegradable compositions that are inserted into an individual. For example, suitable biodegradable compositions comprising a RIP include fibrin sealant. A fibrin sealant comprises concentrated fibrinogen and thrombin and usually other coagulation factors, typically in powdered form. In contact with blood, fibrin sealant immediately forms a clot, making fibrin sealant useful in a wide variety of surgical procedures as a hemostatic agent and as a tissue or wound sealant. See Albala, Cardiovasc. Surg. 11 (Suppl. 1): 5-11. As used herein, a “fibrin sealant” includes such compositions as surgical adhesive glue, surgical sealant, or the like. As with bone cement, fibrin sealants may be manufactured or stored with admixed RIP while in powdered form or in any other pre-solidified or pre-implanted form. A RIP similarly may be added to biodegradable compositions like collagen sheet hydrogels or hydrocolloids or the like, which are used for wound care. Hydrogels and hydrocolloids include collagen alginate wound dressings, temporary skin replacements, and scar removal sheets.

Assay Systems for Determining Activity of RIP and RIP Formulations

The mechanism through which RIP inhibits quorum sensing mechanisms, as discussed above, involves inhibition of the phosphorylation of TRAP. There is evidence of the presence of TRAP and TRAP phosphorylation in S. epidermidis, indicating that there is a similar quorum sensing mechanism both in S. aureus and in S. epidermidis and the potential for RIP to interfere with biofilm formation and infections caused by both species. In addition, there is evidence that TRAP is conserved among all staphylococcal strains and species; therefore, RIP should be effective against any type of Staphylococcus. Further, other infection-causing bacteria appear to have proteins with sequence similarity to TRAP, including Bacillus subtilus, Bacillus anthracis, Bacillus cereus, Listeria innocua, and Listeria monoctogenes. Moreover, RAP is an ortholog of the ribosomal protein L2, encoded by the rplB gene. See Korem et al., FEMS Microbiol. Lett. 223: 167-75 (2003), which is incorporated by reference herein with regard to its description of RAP orthologs encoded by the rplB gene. L2 is highly conserved among bacteria, including Streptococcus spp, Listeria spp, Lactococcus spp, Enterococcus spp, Escherichia coli, Clostridium acetobtylicum, and Bacillus spp. This finding indicates that treatment aimed at disturbing the function of RAP in S. aureus also will be effective in treating L2-synthesizing bacteria as well.

Preferred RNAIII-inhibiting peptides according to the invention directly or indirectly exhibit RNAIII inhibiting activity, which can be assayed using a number of routine screens. RIP inhibits Staphylococcus adherence and toxin production by interfering with the known function of a staphylococcal quorum sensing system. As discussed above, RIP competes with RAP induction of TRAP phosphorylation, leading to the inhibition of TRAP phosphorylation. See Balaban et al., J. Biol. Chem. 276: 2658-67 (2001). This decreases cell adhesion, biofilm formation, and RNAIII synthesis and ultimately suppresses the virulence phenotype. See Balaban et al., Science 280: 438-40 (1998). For example, RIP inhibition of RNAIII production or TRAP phosphorylation can be assayed in vitro using the procedures described in Balaban et al., Peptides 21:1301-11 (2000), incorporated herein by reference. The activity of the amide form of a synthetic RIP analogue YSPWTNF(-NH₂) (the non-amidated form of synthetic RIP is inactive) can be demonstrated in a cellulitis model, using Smith Diffuse mice infected with S. aureus, in a septic arthritis model, testing mice against S. aureus LS-1, in a keratitis model, testing rabbits against S. aureus 8325-4, in an osteomyelitis model, testing rabbits against S. aureus MS, and in a mastitis model, testing cows against S. aureus Newbould 305, AE-1, and environmental infections. See Balaban et al., Peptides 21:1301-11 (2000) and TABLE 1. These findings demonstrate the range of RIP activities and screens available to assay RIP activity and further indicate that RIP prevents and suppresses staphylococcal infections.

TABLE 1

| | | S. aureus | Animals tested (n) | | % animals | |
|---|---|---|---|---|---|---|
| Infection | Model | strain | −RIP | +RIP | disease free | P |
| Osteomyelitis | Rabbit | MS | 7 | 8 | 58 | 0.02 |
| Sepsis | Mouse | LS-1 | 10 | 11 | 44 | 0.04 |
| Arthritis | Mouse | LS-1 | 10 | 10 | 60 | 0.006 |
| Keratitis | Rabbit | 8325-4 | 8 | 8 | 40 | 0.015 |
| Mastitis | Cow | Newbould/AE-1 | 6 | 7 | 70-100 | <0.05 |
| Cellulitis/sepsis | Mouse | Smith diffuse | 22 | 20 | Up to 100 | 0.02 |
| Graft injection | Rat | MRSA, MRSE, | >1000 | >1000 | Up to 100 | <0.05 |
| | | VISA, VISE, | | | | |
| | | GISA, GISE, | | | | |
| | | MSSA, MSSE | | | | |

The screening assay can be a binding assay, wherein one or more of the molecules may be joined to a label that provides a detectable signal. Alternatively, a screening assay can determine the effect of a candidate RIP on RNAIII production and/or virulence factor production. For example, the effect of the candidate peptide on rnaiii transcription in Staphylococcus can be measured. Such screening assays can utilize recombinant host cells containing reporter gene systems such as CAT (chloramphenicol acetyltransferase), β-galactosidase, and the like, according to well-known procedures in the art. Alternatively, the screening assay can detect rnaiii or virulence factor transcription using hybridization techniques that also are well known in the art. Purified RIP further may be used to determine a three-dimensional crystal structure, which can be used for modeling intermolecular interactions.

In vitro High Throughput Analysis of RIP Formulations

The following screening assay for RIP compositions exemplifies the types of assays that may be used to determine whether a particular RIP or RIP composition or formulation exhibits the desired level of biological activity. In this assay system, agr expression is tested in a high throughput assay using an RNAIII reporter gene assay, which is confirmed by Northern blotting. S. aureus cells in early exponential growth (about 2×10⁷ colony forming units (CFU)) containing the rnaiii::blaZ fusion construct are grown with increasing concentrations of the RIP formulations in 96 well plates at 37° C. with shaking for 2.5-5 hrs. The rnaiii::blaZ fusion construct is described in Gov et al., 2001. In this assay, β-lactamase acts as a reporter gene for RNAIII expression. Bacterial viability is tested by determining O.D. 650 nm and further by plating to determine CFU. β-lactamase activity is measured by adding nitrocefin, a substrate for β-lactamase. Hydrolysis of nitrocefin by β-lactamase is indicated by a change in relative adsorption at 490 nm and 650 nm, where yellow color indicates no RNAIII synthesis, and pink color indicates RNAIII synthesis.

Formulations showing efficacy in the high throughput assay may be confirmed by Northern blotting. Bacteria similarly are grown with candidate RIP formulations. Cells are then collected by centrifugation, and total RNA is extracted and separated by agarose gel electrophoresis. RNAIII is detected by hybridization to radiolabeled RNAIII-specific DNA produced by PCR, for example. Control formulations containing random peptides typically are tested at 0-10 μg/10⁷ bacteria.

In vivo Analysis of RIP Formulations

Candidate peptides also can be assayed for activity in vivo, for example by screening for an effect on Staphylococcus virulence factor production in a non-human animal model. The candidate peptide is administered to an animal that has been infected with Staphylococcus or that has received an infectious dose of Staphylococcus in conjunction with the candidate peptide. The candidate peptide can be administered in any manner appropriate for a desired result. For example, the candidate peptide can be administered by injection intravenously, intramuscularly, subcutaneously, or directly into the tissue in which the desired affect is to be achieved, or the candidate can be delivered topically, orally, etc. The peptide can be used to coat a device that will then be implanted into the animal. The effect of the peptide can be monitored by any suitable method, such as assessing the number and size of Staphylococcus-associated lesions, microbiological evidence of infection, overall health, etc.

The selected animal model will vary with a number of factors known in the art, including the particular pathogenic strain of Staphylococcus or targeted disease against which candidate agents are to be screened. A rat graft model is especially useful when assessing the ability of a formulation to suppress infections associated with biofilm formation. Giacometti et al., Antimicrob. Agents Chemother. 47: 1979-83 (2003); Cirioni et al., Circulation 108: 767-71 (2003); Balaban et al., J. Infect. Dis. 187: 625-30 (2003). This model is highly relevant to the clinical setting because it provides a time interval between bacterial challenge and biofilm infection, typically within 72 hours, allowing testing of the optimal route of administration and dose of the RIP formulation. This model provides a challenging test of RIP activity because biofilms are known to be extremely resistant to antibiotics.

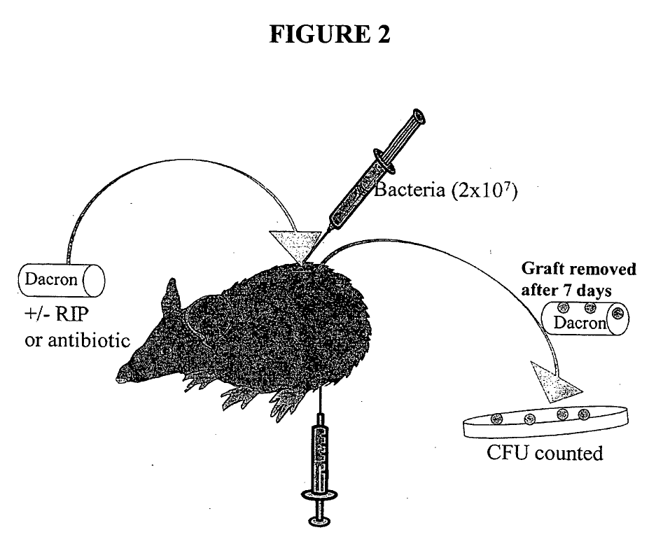

The typical steps in a rat graft model are shown in FIG. 2. Using this test, RIP was shown to reduce infection by four orders of magnitude when grafts were soaked with 20 μg/mL RIP for 20 minutes or when RIP was injected by an intraperitoneal route at 10 mg RIP/kg body weight. In a typical experiment, Wistar adult male rats (n=10) are anesthetized, and a subcutaneous pocket is made on each side of the median line by a 1.5 cm incision. Sterile collagen-sealed double velour knitted polyethylene terephthalate (Dacron) grafts (1 cm²) (Albograft™, Italy) are soaked with saline, a random peptide having no RIP activity, or a RIP and then implanted into the pockets. Pockets are closed with skin clips, and 2×10⁷ CFU/mL bacteria are inoculated onto the graft surface using a tuberculin syringe to create a subcutaneous fluid-filled pocket. The animals are returned to individual cages and examined daily. Animals receive an intravenous or oral administration of RIP or a RIP formulation 0-6 days after the graft infection. Free RIP is administered via an intraperitoneal route as a positive control. Grafts are explanted at 7 days following implantation, and CFU are determined according to known procedures, e.g., Giacometti et al. (2003). The explanted grafts are placed in sterile tubes, washed in sterile saline solution, placed in tubes containing 10 mL of phosphate-buffered saline solution, and sonicated for 5 minutes to remove the adherent bacteria from the grafts. After sonication, grafts are microscopically checked to verify that all bacteria are removed. No significant differences in cell viability (CFU/mL) were present upon testing the effect of sonication for up to 10 minutes on either antibiotic sensitive or antibiotic resistant bacteria. Viable bacteria are quantified by culturing serial dilutions (0.1 mL) of the bacterial suspension on blood agar plates. All plates are incubated at 37° C. for 48 hours and evaluated for number of CFUs per plate. The limit of detection for this method is approximately 10 CFU/mL.

A special modification of the rat graft assay may be used particularly to assay the effectiveness of RIP compositions administered with bone cement compositions. In this version of the assay, a bone cement composition substitutes for the Dacron graft. The bone cement may be injected or implanted into the test rat, in which case the RIP may be formulated with the bone cement composition, or the bone cement composition may be hardened and inserted into the rat's subcutaneous pocket, in which case the bone cement may be soaked with RIP prior to insertion. In either case, a RIP composition also may be applied, for example, as a sustained release formulation at the site of bone cement injection or insertion. As with the Dacron model, a RIP composition alternatively or additionally may be delivered intravenously or orally 0-6 days after the bone cement injection or insertion. Free RIP is administered via an intraperitoneal route as a positive control, as before. At day 7, the bone cement is surgically removed and assayed for infection or biofilm formation by the method described in either Van de Belt et al., Acta Orthop. Scand. 71: 625-29 (2000) or Neut et al., Acta Orthopaedica 76: 109-11 (2005), or the like. The rat graft model is used to test various routes of RIP and bone cement administration to be used in clinical practice.

Methods of Administering a RIP Composition

The present invention provides a method of administering a bone cement composition that also comprises administering a RIP composition, where the RIP composition may added before, during or after addition of the bone cement or may be admixed with the bone cement. When RIP is administered before the bone cement, RIP is still present in an amount effective to treat or reduce the risk of bacterial infection at the time the bone cement is implanted. When RIP is administered concurrently or shortly after implanting the bone cement, RIP is used to treat or reduce the risk of an infection arising from administering the bone cement, i.e., an infection associated with the implanting of the bone cement. The RIP composition may further comprise an antimicrobial agent, such as an antibiotic or antimicrobial peptide, or an anesthetic. The administration of the bone cement composition comprising RIP may be repeated on the same individual, as necessary.

The term “treatment” or “treating” means any therapeutic intervention in an individual animal, e.g., a mammal, preferably a human. Treatment includes (i) “prevention,” causing the clinical symptoms not to develop, e.g., preventing infection from occurring and/or developing to a harmful state; (ii) “inhibition,” arresting the development of clinical symptoms, e.g., stopping an ongoing infection so that the infection is eliminated completely or to the degree that it is no longer harmful; and (iii) “relief,” causing the regression of clinical symptoms, e.g., causing a relief of fever and/or inflammation caused by an infection. Treatment may comprise the prevention, inhibition, or relief of biofilm formation. Administration to an individual “at risk” of having a bacterial infection means that the individual has not necessarily been diagnosed with a bacterial infection, but the individual's circumstances place the individual at higher than normal risk for infection of infection, e.g., the individual is a recipient of a bone cement composition. Administration to an individual “suspected” of having a bacterial infection means the individual is showing some initial signs of infection, e.g., elevated fever, but a diagnosis has not yet been made or confirmed.

The term “effective amount” means a dosage sufficient to provide treatment or prophylaxis. The quantities of active ingredients necessary for effective therapy will depend on many different factors, including means of administration, target site, physiological state of the patient, and other medicaments administered; therefore, treatment dosages should be titrated to optimize safety and efficacy. Typically, dosages used in vitro may provide useful guidance in the amounts useful for in vivo administration of the active ingredients. Animal testing of effective doses for treatment of particular disorders will provide further predictive indication of human dosage. The concentration of the active ingredients in the pharmaceutical formulations typically vary from less than about 0.1%, usually at or at least about 2% to as much as 25% to 50% or more by weight, and will be selected primarily by fluid volumes, viscosities, etc., in accordance with the particular mode of administration selected. A preferred range of weight percent of RIP in the present compositions varies from about 7% to about 25%. Various appropriate considerations are described, for example, in Goodman and Gilman, “The Pharmacological Basis of Therapeutics,” Hardman et al., eds., 10^{th} ed., McGraw-Hill, (2001) and “Remington: The Science and Practice of Pharmacy,” University of the Sciences in Philadelphia, 21st ed., Mack Publishing Co., Easton Pa. (2005), both of which are herein incorporated by reference with respect to effective dosages for various pharmaceutical formulations and for methods for administration discussed therein, including administration by oral, intravenous, intraperitoneal, intramuscular, transdermal, nasal, topical, and iontophoretic routes, and the like. Such routes of RIP administration are contemplated herein, where RIP is not solely administered as a component of the bone cement composition or other biodegradable composition.

For the purpose of the invention, a “RIP composition” comprises an RNAIII-inhibiting peptide and possibly other pharmacologically active agents. Suitable active agents include antibiotics and antimicrobial peptides, which are described further below. Useful antibiotics include, but are not limited to, an amino-glycoside (e.g., gentamycin), a beta-lactam (e.g., penicillin), or a cephalosporin. The present invention advantageously allows antibiotics to be used with a lower risk of inducing resistance. Bacteria do not appear to become resistant to RIP itself, even after prolonged exposure. Further, RIP reduces biofilm, particularly the protective environment of the glycocalyx, which contributes to increasing resistance of periprosthetic infections to chemotherapy. The concomitant reduction in the risk of inducing resistance promotes the use of antibiotics that otherwise would be reserved as a last resort. Vancomycin is one such antibiotic of last resort; however, after nearly 40 years of use, resistant strains are becoming more prevalent. In the present invention, RIP potentiates the activity of vancomycin, minimizing the risk that more vancomycin-resistant strains will arise. For this reason, it is believed that the present invention will significantly prolong the effectiveness of antibiotics of last resort.

Active agents may be administered to the individual in the same composition as the RIP or in a separate formulation at or around the same time as the RIP composition is administered. For example, the present method comprises oral co-administration of antibiotics with bone cement compositions comprising RIP. Administration of the RIP and antibiotic may occur within about 48 hours, preferably within about 2-8 hours and, most preferably, substantially concurrently with the administration of the bone cement or other biodegradable composition.

Antimicrobial Peptides

As described above, the compositions according to the present invention may comprise an antimicrobial peptide. Antimicrobial peptides are an important component of the innate immune response in most multi-cellular organisms, which represents a first line of host defense against an array of microorganisms. Antimicrobial peptides have a broad spectrum of activities, killing or neutralizing both gram-negative and gram-positive bacteria, including antibiotic-resistant strains. See Hancock, Lancet Infect. Dis. 1: 156-64 (2001). Wang, University of Nebraska Medical Center, Antimicrobial Peptide Database, at http://aps.unmc.edu/AP/main.php (last modified Mar. 5, 2005), which is incorporated herein by reference in its entirety, provides a database of about 500 antimicrobial peptides with antibacterial activity that potentially are useful for the present invention. Antimicrobial peptides usually are made up of between 12 and 50 amino acid residues and are polycationic. Usually about 50% of their amino acids are hydrophobic and they are generally amphipathic, where their primary amino acid sequence comprises alternating hydrophobic and polar residues. Antimicrobial peptides fit into one of four structural categories: (i) P-sheet structures that are stabilized by multiple disulfide bonds (e.g., human defensin-1), (ii) covalently stabilized loop structures (e.g., bactenecin), (iii) tryptophan (Trp)-rich, extended helical peptides (e.g., indolicidin), and (iv) amphipathic a-helices (e.g., the magainins and cecropins). See Hwang et al., Biochem. Cell Biol. 76: 235-46 (1998); Stark et al., Antimicrob. Agents Chemother 46: 3585-90 (2002).

RIP Carrier Systems

In one embodiment, a RIP composition is in a carrier system. Carrier systems may allow sustained release of RIP in and/or around the bone cement implant. Nanoparticles provide a preferred RIP carrier system, as do liposomes, described below. Nanoparticles typically comprise either a polymeric matrix (“nanospheres”) or a reservoir system comprising an oily core surrounded by a thin polymeric wall (“nanocapsules”), where the core comprises the RIP composition. Polymers suitable for the preparation of nanoparticles include poly(alkylcyanoacrylates), and polyesters such as poly(lactic acid) (PLA), poly(glycolic acid), poly(-caprolactone) and their copolymers.

Nanoparticle size and morphology may be altered, as well, to yield formulations with desired physicochemical characteristics, loading, and controlled release properties appropriate for a RIP composition. By modifying the formulation appropriately, it is possible to mediate a burst release of RIP for the rapid onset of its antibacterial effects. “Burst release kinetics” here means that most of the RIP is released from the formulation within 24 hours, preferably within 1-7 hours, after the RIP composition is administered to a host.

Nanoparticles may be fabricated using biodegradable polyesters, e.g., polymers of poly(lactic acid) (PLA) and copolymers that are manufactured with varying quantities of glycolic acid (PLGA). PLA is more hydrophobic in comparison to PLGA; therefore, PLA offers a relatively extended release profile. Similarly, the ratio of glycolic acid to lactic acid in the copolymerization process effects the degradative properties of the resultant copolymer. In one embodiment, low molecular weight (14 kDa) PLGA is copolymerized with a high (50%) glycolide content (PLGA 50:50). These particles will degrade comparatively rapidly due to the low molecular weight and high glycolide content of the PLGA used. It is expected that 90% of the RIP will be released within 30 days, and 90% resorption of the polymer will occur within 5 weeks. To obtain nanospheres with an intermediate or long degradation profile, the aforementioned formulation may comprise a higher molecular weight copolymer (e.g., 60-100 kDa), with or without a lower glycolide content (PLGA 65:35 or 75:25). In short, a comprehensive range of PLA and PLGA polymer molecular weight, lactic/glycolic acid ratios, and PLA-PLGA blends may be used to optimize loading and release profiles.

RIP compositions may be associated with the nanospheres either by encapsulation, adsorption onto the particle surface, or both. Depending on the particular molecules in the RIP composition, peptide loading efficiencies of up to 100% are expected when a 10% w/w loading level is attempted. From previous encapsulation studies, an increase in drug loading is expected to increase in particle size; therefore, high and low peptide loading formulations may be used with large (˜2000-5000 nm average diameter) and small (˜200-500 nm average diameter) particle sizes, respectively. Note that the larger size particles are considered “nanoparticles” for the purpose of the invention, even though their diameters may exceed a micron.

Compositions Comprising RIP

Formulations comprising RIP are known and described, for example, in U.S. Pat. No. 6,291,431, application Ser. No. 10/358,448, filed Feb. 3, 2003, and application Ser. No. 09/839,695, filed Apr. 19, 2001, which are incorporated by reference herein. When RIP is formulated in a bone cement composition, care must be taken that the components of the RIP composition do not interfere with the setting of the bone cement. The effect of the components of an admixed RIP composition on bone cement polymerization can be tested in vitro. Likewise, the effect of the setting of bone cement on the activity of RIP can be tested in vitro using any of the procedures described above or by use of animal models, discussed further below. Methods of combining the use of RIP and bone cement can be adjusted to prevent the loss of RIP activity. For example, the RIP composition may be contained within a carrier system or injected after the bone cement has hardened, as described elsewhere herein.

The concentration of RIP in any formulation may be varied to provide the optimum therapeutic response. For example, several divided doses may be administered daily or the dose may be proportionally reduced as indicated by the therapeutic situation. Human dosage levels for treating infections are known and generally include a daily dose from about 0.1 to 500 mg/kg of body weight per day, preferably about 6 to 200 mg/kg, and most preferably about 12 to 100 mg/kg. The amount of formulation administered will depend, of course, on the subject and the severity of the affliction, the manner and schedule of administration and the judgment of the prescribing physician. When administered intravenously, for example, serum concentrations can be maintained at levels sufficient to treat infection in less than 10 days, although an advantage offered by the present invention is the ability to extend treatment for longer than 10 days at relatively low levels of the RIP composition because of the decreased likelihood that bacteria will develop resistance to the present composition over a long course of treatment.

Pharmaceutical grade organic or inorganic carriers or diluents can be used to make up compositions containing the therapeutically active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. The compositions may include other pharmaceutical excipients, carriers, etc. Suitable excipients are water, saline, dextrose, glycerol, ethanol or the like. Methods of preparing pharmaceutical compositions are well known to those skilled in the art. See, for example, “Remington: The Science and Practice of Pharmacy,” University of the Sciences in Philadelphia, 21st ed., Mack Publishing Co., Easton Pa. (2005), incorporated by reference herein. As described above, the effect of any of these components on bone cement setting first can be tested in vitro.

The RIP compositions of the invention may be administered in a variety of unit dosage forms depending on the method of administration. For example, unit dosage forms suitable for oral administration include solid dosage forms such as powder, tablets, pills, and capsules, and liquid dosage forms, such as elixirs, syrups, and suspensions. The active ingredients may also be administered parenterally in sterile liquid dosage forms. Gelatin capsules contain the active ingredient and as inactive ingredients powdered carriers, such as glucose, lactose, sucrose, mannitol, starch, cellulose or cellulose derivatives, magnesium stearate, stearic acid, sodium saccharin, talcum, magnesium carbonate and the like.

Examples of inactive ingredients that may be added to the composition of the invention include agents that provide desirable color, taste, stability, buffering capacity, dispersion or other features, such as red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The RIP compositions of the invention may also be administered via liposomes, which include emulsions, foams, micelles, insoluble monolayers, liquid crystals, phospholipid dispersions, lamellar layers and the like. In these preparations the composition of the invention to be delivered may be incorporated as part of the liposome, alone or in conjunction with a targeting molecule, such as antibody, or with other therapeutic or immunogenic compositions. Thus, liposomes comprising a desired composition of the invention can delivered systemically or can be directed to a tissue of interest.

Liposomes for use in the invention are formed from standard vesicle-forming lipids, which generally include neutral and negatively charged phospholipids and sterols such as cholesterol. The selection of lipids is generally guided by the desired liposome size, acid lability and stability in the blood stream. A variety of methods are available for preparing liposomes as described in Szoka et al., Ann. Rev. Biophys. Bioeng. 9: 467 (1980), U.S. Pat. Nos. 4,235,871, 4,501,728, 4,837,028, and 5,019,369, which are incorporated herein by reference. A liposome suspension containing a composition of the invention may be administered intravenously, locally, topically, etc. in a dose that varies according to the manner of administration, the composition of the invention being delivered, and the stage of the disease being treated, among other things.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95%, more preferably 25%-75%, of a RIP. RIP compositions of the invention additionally can be delivered in a depot-type system, an encapsulated form, or an implant by techniques well-known in the art. For example, a RIP composition can be administered in a biogradable matrix or foam at a site where the bone cement is to be inserted, thereby assuring that the RIP composition is exposed to all the tissues surrounding the bone cement. Similarly, the RIP composition can be delivered via a pump, e.g. an osmotic pump, to a tissue of interest.

For aerosol administration, the compositions of the invention are preferably supplied in finely divided form along with a surfactant and propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

### EXAMPLES

1. Sustained Release of RIP from PMMA Beads Loaded with RIP.

1.1 Preparation of PMMA Beads:

PMMA beads were prepared by adding RIP and/or vancomycin powder to Surgical Simplex P® radio-opaque bone cement (Stryker Orthopedics, Mahwah, N.J.). The bone cement was prepared by mixing 3 g PMMA, 15 g methyl methacrylate-styrene-copolymer and 2 g barium sulfate in powder form with 9.75 mL methyl methacrylate, 0.25 mL N,N-dimethyl-paratoluidine, with 75±15 ppm hydroquinone.

Beads containing 7.5%, 15%, and 22.5% (w/w) RIP were prepared by mixing 150 mg, 200 mg, and 340 mg RIP powder with 1850 mg, 1132 mg, and 1170 mg of cement powder, respectively. 0.925 mL, 0.626 mL, or 0.585 mL of methyl methacrylate liquid monomer, respectively, were added to the RIP/cement powder compositions. Beads containing 15% (w/w) vancomycin were prepared by mixing 200 mg of vancomycin and 1132 mg of cement powder, then adding 0.626 mL of methyl methacrylate liquid monomer. Beads containing 7.5% vancomycin and 15% RIP (w/w) were prepared by mixing 150 mg of vancomycin, 300 mg RIP and 1550 mg of cement powder, then adding 0.775 mL of methyl methacrylate liquid monomer. Powder mixes were blended with a spatula to form putty like material. The cement was spread in a sterile bead mold and allowed to polymerize for 2 hours. The polymerized beads had a 3 mm diameter and weighed 25 mg. Beads were stored at −70° C.

1.2 Release of RIP from PMMA Beads:

The continuous flow chamber described by Perry et al., Clin. Orthoped. Related Res. 403: 49-53 (2002) was used to test the release of RIP from the beads. Individual beads were placed in 5 mL test tubes (Becton Dickinson Labware, Franklin Lakes, N.J.) containing 1 mL Krebs ringer buffer (KRB) and held at 37° C. with a Thermolyne Type Dri-bath (Bamstead/Thernolyne, Model 0817615, Dubuque, Iowa). A KRB solution at 37° C. was pumped at 1 mL/h through the test tube, and the eluate was collected in a fraction collector. Samples were collected hourly for 24 h and then every 2 h over the next 24 h. Samples were stored at −70° C. until tested for the presence of RIP.

1.3 Determination of the RIP Concentration:

Soluble material released from the beads at different time points (1-48 h) from three different 7.5% RIP-loaded beads (run A, B and C) and from pulverized PMMA beads was applied to a reverse phase C18 column (Hypersil GOLD, Thermo Electron Corp.) at a flow rate of 1 mL/min with a mobile phase containing 0.1% trifluoroacetic acid (TFA) in water. RIP was eluted using a gradient of acetonitrile (ACN) from 0-70% for 15 min; RIP eluted from the column in 54% ACN. The concentration of RIP eluted from the reverse phase column was determined by the adsorption of the eluted RIP.

1.4 Release Profile of RIP:

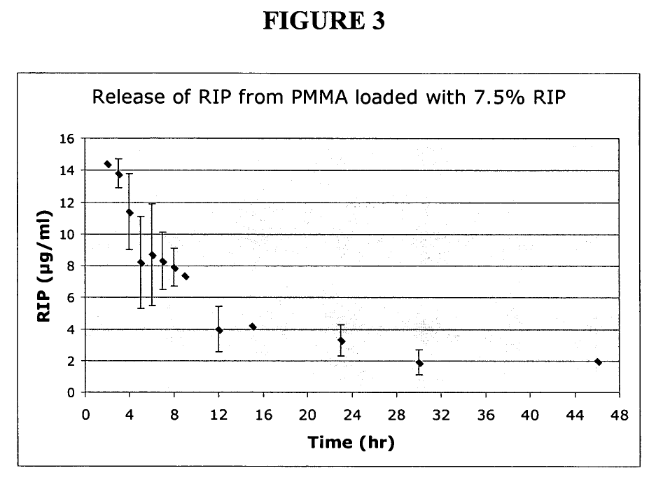

The continuous flow system with HPLC analysis of RIP was used to test the release profile of RIP from PMMA beads that were loaded with 7.5% RIP. As shown in FIG. 3, release profiles were bimodal with initial rapid release of RIP over about 12 hours, followed by sustained, slow release of RIP over at least about 48 hours. The experiment demonstrates that RIP may be released over a sustained period from a bone cement composition comprising RIP.

2. In vivo Efficacy of PMMA Beads Loaded with RIP and/or Vancomycin.

2.1 Animal Model Experiments:

PMMA beads containing RIP and/or vancomycin were prepared as described in Example 1.1, above. Six groups of adult male Wistar rats (200-300 g) were then tested by inserting a bead chosen from one of following groups: (1) PMMA beads (8 rats); (2) 7.5% RIP-loaded PMMA beads (5 rats); (3) 7.5% vancomycin-loaded PMMA beads (6 rats); (4) 15% RIP-loaded PMMA beads (10 rats); (5) 22.5% RIP-loaded PMMA beads (10 rats); and (6) 15% RIP plus 7.5% vancomycin-loaded PMMA beads (7 rats).

Rats were anesthetized with ether, the hair of the back was shaved, and the skin was cleaned with a 10% povidone-iodine solution. A subcutaneous pocket was made on each side of the median line by a 1.5 cm incision. A bead from one of the groups of beads listed above was aseptically implanted into the pockets using sterile surgical forceps, and the pockets were closed using skin clips. 1 mL of saline containing 2×10⁷ CFU methicillin-resistant S. aureus, ATCC 43300, was inoculated around the PMMA bead surface using a tuberculin syringe to create a subcutaneous fluid-filled pocket. The animals were returned to individual cages and examined daily. Beads were explanted seven days following implantation.

2.2 Assessment of Infection:

The tissues surrounding the PMMA beads were aseptically dissected using sterile lancets. The beads were removed with sterile forceps, placed in sterile tubes, and soaked in 2 mL of sterile saline solution for 5 minutes to remove non-adherent organisms. Beads were then placed in tubes containing 10 mL of phosphate-buffered saline solution and sonicated for 5 min to remove the adherent bacteria from the surfaces. Aliquots of 0.1 mL were serially diluted in 9.9 mL of 10 mM of sodium HEPES buffer (pH 7.2) (Sigma-Aldrich, Milan, Italy). Viable bacteria were quantified by plating 0.1 mL of each dilution on blood agar plates, which were incubated at 37° C. for 48 h to detect CFUs. Organisms were quantified by counting the number of CFUs per plate. The limit of detection for this method was 10 CFU/mL.

2.3 Statistical Analysis:

Statistical analysis was performed using SPSS 11.5 for Windows package (SPSS Inc., Ill.). Quantitative results for the in vivo experiments are presented as mean±standard deviation. Results were compared by nonparametric tests using Kruskal-Wallis and Mann-Whitney U for comparisons between groups.

2.4 Prevention of Infection:

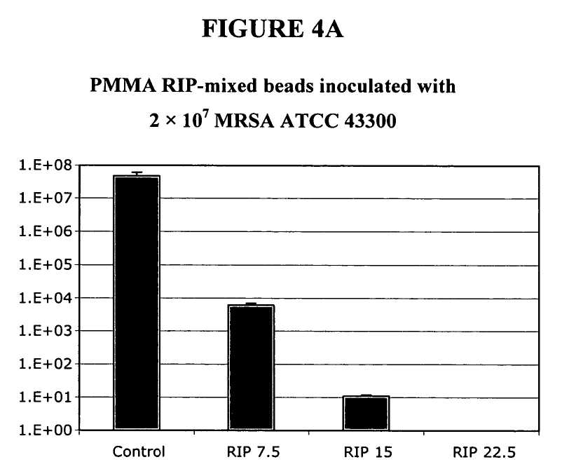

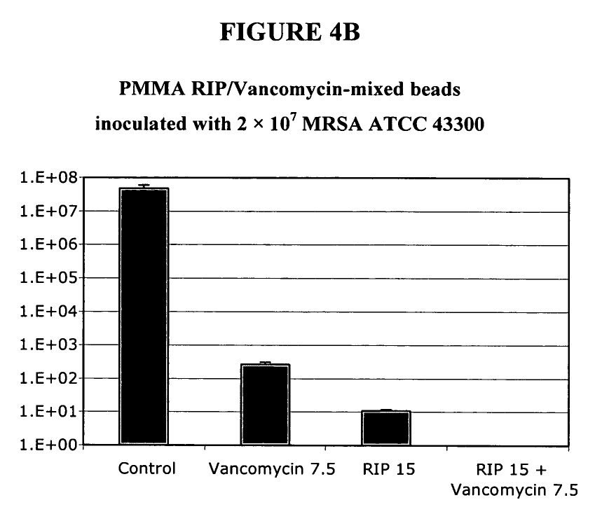

PMMA beads loaded with RIP and/or vancomycin were tested in vivo for the formation of S. aureus biofilm. As shown in FIG. 4A, while control animals yielded 4.8±1.2×10⁷ CFU/mL, PMMA loaded with 7.5% RIP yielded 6.1±0.9×10³ CFU/mL, PMMA loaded with 15% RIP yielded 1.1±0.1×10¹ CFU/mL, and those loaded with 22.5% RIP yielded <10 CFU/mL. In FIG. 4A, CFU/mL are plotted against RIP loaded at the specified weight percent. PMMA beads loaded with 22.5% RIP reduced infection by 7 orders of magnitude in this assay. As shown in FIG. 4B, PMMA loaded with 7.5% vancomycin yielded 2.7±0.4×10² CFU/mL, and those loaded with 15% RIP and 7.5% vancomycin yielded <10 CFU/mL. Thus, RIP potentiates the effect of vancomycin.

In summary, all treatment groups were more active than the control (p≦0.001). Among loaded beads, the combination of vancomycin plus RIP was more active than vancomycin alone (p≦0.001). Beads with 15% RIP showed more activity than beads containing 7.5% vancomycin or RIP (p≦0.001). Beads containing 7.5% vancomycin showed more activity than those containing 7.5% RIP (p≦0.004). Beads containing 22.5% RIP or 15% RIP plus 7.5% vancomycin yielded results below the limit of detection of the technique (<10 CFU), and showed more activity than all other combinations studied (p≦0.003).

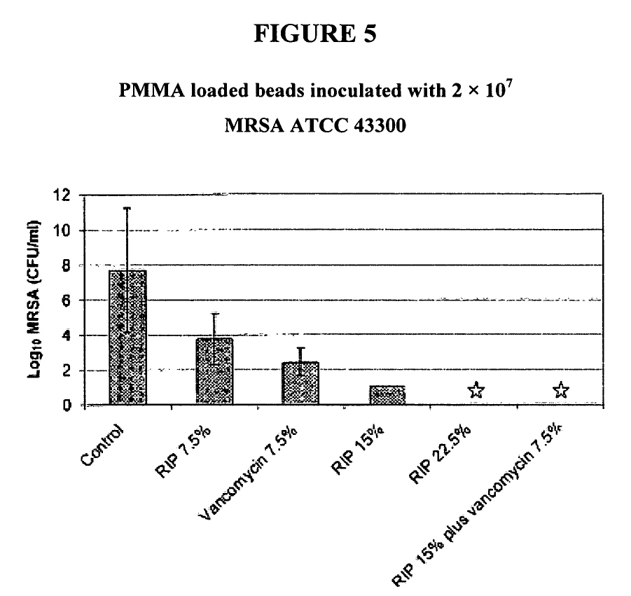

FIG. 5 shows the results of another experiment performed under the same conditions as those described in FIG. 4. RIP clearly displayed efficacy in vivo when administered in a bone cement composition for a week. The star over the columns for 22.5% RIP and 15% RIP plus vancomycin indicate that bacterial infection was statistically undetectable within the limit of error in the assay.

These experiments demonstrate that RIP loaded in PMMA is biologically active in vivo for an extended period, one week in these studies. In particular, PMMA beads loaded with 22.5% RIP alone or 15% RIP plus 7.5% vancomycin totally prevented staphylococcal biofilm formation in vivo for the duration of the study.

All publications and patents mentioned herein are incorporated herein by reference to disclose and describe the specific methods and/or materials in connection with which the publications and patents are cited. The publications and patents discussed herein are provided solely for their disclosure prior to the filing date of the present application. Nothing herein is to be construed as an admission that the present invention is not entitled to antedate such publication or patent by virtue of prior invention. Further, the dates of publication or issuance provided may need to be independently confirmed.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A composition comprising a bone cement composition and an RNAIII-inhibiting peptide (RIP) in an effective amount to treat or reduce the risk of bacterial infection in a mammalian individual receiving said composition.

2. The composition of claim 1, where RIP represents at least about 7.5% w/w of the composition.

3. The composition of claim 2, where RIP represents at least about 15% w/w of the composition.

4. The composition of claim 3, where RIP represents at least about 25% w/w of the composition.

5. The composition of claim 1, further comprising an antibiotic or antimicrobial peptide.

6. The composition of claim 5, where the antibiotic is vancomycin or daptomycin.

7. The composition of claim 5, where the antibiotic is an amino-glycoside or a beta-lactam.

8. The composition of claim 1, further comprising a bone morphogenic protein.

9. The composition of claim 1, further comprising an anesthetic.

10. The composition of claim 1, where the bone cement composition comprises polymethylmethacrylate or methyl methacrylate.

11. The composition of claim 1, where the bone cement is an injectable ceramic cement, an injectable calcium phosphate hydraulic cement, a calcium deficient hydroxyapatite cement, a dahllite cement, or a brushite cement.

12. The composition of claim 1, where the bone cement composition comprises a component in powdered form.

13. The composition of claim 1, where the RIP comprises:
(a) five contiguous amino acids of the sequence YX₂PX₁TNF, where X₁ is C, W, I or a modified amino acid, and X₂ is K or S; or
(b) amino acids having a sequence that differs from the sequence YX₂PX₁TNF by two substitutions or deletions, where X₁ is C, W, I or a modified amino acid, and X₂ is K or S.

14. The composition of claim 1, where the RIP is formulated in a carrier system.

15. The composition of claim 14, where the carrier system comprises nanoparticles.

16. A method of administering a bone cement composition to an individual, comprising inserting into the individual a bone cement composition comprising an RNAIII-inhibiting peptide (RIP) composition, where the RIP is in an amount effective to treat or reduce the risk of bacterial infection in the individual.

17. The method of claim 16, further comprising administering an antibiotic to the individual.

18. The method of claim 17, where the antibiotic is vancomycin or daptomycin.

19. The method of claim 17, where said bone cement composition comprises the antibiotic.

20. The method of claim 16, where the bone cement composition comprises said effective amount of RIP for at least about seven days after said inserting.

21. The method of claim 16, where said amount effective of RIP is capable of reducing a bacterial load in the individual by at least about five, six, or seven orders of magnitude.

22. The method of claim 16, where the RIP is admixed with a powdered component of the bone cement composition prior to administration of the bone cement.

23. The method of claim 16, where the RIP is admixed with the bone cement prior to the setting of the bone cement.

24. The method of claim 16, further comprising administration of a second RIP composition before, concomitantly, or after said inserting.

25. A method of treating or reducing the risk of bacterial infection in an individual, comprising administering a RIP composition to the individual in an amount effective to treat or reduce the risk of infection, where the infection is associated with bone cement inserted in the individual.

26. The method of claim 25, where the RIP composition is administered concurrently with the insertion of the bone cement composition in the individual.

27. The method of claim 25, where the RIP composition is administered after the bone cement was implanted in the individual.

28. The method of claim 25, comprising parenterally administering the RIP composition.

29. The method of claim 25, comprising orally administering the RIP composition.

30. The method of claim 25, where the RIP composition is a formulation capable of burst-release kinetics.

31. The method of claim 25, where the RIP composition is a formulation capable of sustained release.
